(19) United States
(12) Patent Application Publication
(10) Pub. No.: US 2026/0240785 A1
(43) Pub. Date: Aug. 20, 2026

(54) ANGIOGENESIS INHIBITORS COUPLED TO EMBOLIC MICROPARTICLES

(71) Applicant: Jeffrey C. MAI, Arlington, VA (US)

(72) Inventor: Jeffrey C. MAI, Arlington, VA (US)

(21) Appl. No.: 19/160,630

(22) PCT Filed: Feb. 28, 2024
(86) PCT No.: PCT/US2024/017605

§ 371 Date: Aug. 28, 2025

Related U.S. Application Data
(60) Provisional application No. 63/448,710, filed on Feb. 28, 2023.

(51) Int. Cl. A61K 9/50; A61K 9/00; A61K 39/44

(52) U.S. Cl. CPC A61K 9/5015; A61K 9/0019; A61K 39/44

(57) ABSTRACT

A method of treating a solid tumor or subdural hematoma in a subject in need thereof is described. The method includes administering a therapeutically effective amount of biocompatible polymeric microparticles comprising an angiogenesis inhibitor to the solid tumor using targeted endovascular administration.

### CROSS-REFERENCE TO RELATED APPLICATIONS

This application claims priority to U.S. Provisional Patent Application Ser. No. 63/448,710, filed Feb. 28, 2023, the disclosure of which is incorporated herein by reference.

### BACKGROUND

Avastin is a potent inhibitor of angiogenesis, by inhibiting vascular endothelial growth factor (VEGF). For example, endovascular super-selective administration of Avastin would provide the means to treat solid tumors in the intracranial and extracranial space, as well as other organs in the body. It also may potentially address the problematic recurrence of subdural hematomas (SDH) in neurosurgery, which recur by means of neo-vascularization. Suzuki et al., Neurosurg Rev., 39(3):525-9 (2016).

The incidence of chronic SDH is rising; SDH is protected to become the most common cranial neurosurgical condition among adults by the year 2030. Chronic SDH are currently treated by devascularizing the dura mater with glue, ONYX, or microparticles such as PVA or embospheres. The goal of these methods is to block the arterial supply from the middle meningeal artery (MMA) by physical occlusion of these vessels. This is now the fastest growing new area of endovascular neurosurgery. Joyce et al., Neurosurg Focus, 49(4):E5 (2020). It has supplanted the use of trephination (burr hole placement) for the evacuation of subdural hematomas at our hospital.

However, there is a significant recurrence rate following middle meningeal artery (MMA) embolization, with recurrent bleeding, in spite of efforts to prevent neo-vascularization of the membranes that are present in chronic subdurals. As a result, following MMA embolization, patients require multiple images, prolonged hospitalizations, and repeated surgeries. As the population ages, the cohort of chronic subdurals and their complications as a burden to the health care system continues to grow.

### SUMMARY

The inventors have provided a formulation of Avastin-impregnated gel/microparticles that can be selectively delivered to the MMA or solid tumors by endovascular technique. The targeted delivery to the vascular bed of interest prevents non-target side effects (such as wound breakdown), and enables durable, slow release of Avastin to prevent undesired angiogenesis. This method has widespread utility in the minimally invasive treatment of chronic subdural hematomas and potentially solid tumors, while increasing safety and efficacy of our treatments.

The inventors hypothesized that VEGF-A mediated expression supplied from the MMA vascular bed leads to neovascularization of membranes in chronic SDH. Accordingly, inhibiting VEGF-A using an angiogenesis inhibitor such as Avastin should decrease neovascularization in chronic SDH membranes. By halting neovascularization in chronic SDH, one may anticipate preventing the microhemorrhages and rebleeds that lead to progressive chronic SDH expansion and recurrence. VEGF-inhibition couples with MMA embolization by microparticles may therefore be synergistic in enhancing the therapeutic effectiveness of angiogenesis inhibition in chronic SDH therapy.

In one aspect, the present invention provides a method of treating a solid tumor in a subject in need thereof. The method includes administering a therapeutically effective amount of biocompatible polymeric microparticles comprising an angiogenesis inhibitor to the solid tumor using targeted endovascular administration. In some embodiments, the angiogenesis inhibitor is a vascular endothelial growth factor A (VEGF-A) antagonist, such as Avastin®.

In some embodiments, the solid tumor is a brain tumor. In further embodiments, the brain tumor is a meningioma. In additional embodiments, the solid tumor is also treated by chemotherapy and/or radiotherapy. In yet further embodiments, the biocompatible polymeric microparticles are administered to the middle meningeal artery.

In some embodiments, the microparticles comprise a biocompatible polymer that is a biodegradable polymer. In further embodiments the biocompatible polymer is poly-vinyl alcohol. In some embodiments, the microparticles have a diameter from about 40 μm to about 1200 μm. In further embodiments, the microparticles have a diameter from about 45 μm to about 250 μm. In some embodiments, the angiogenesis inhibitor is distributed within the microparticle. In further embodiments the angiogenesis inhibitor is coated or conjugated to the surface of the microparticle.

Another aspect of the invention provides a method of treating a subdural hematoma in a subject in need thereof. The method includes administering a therapeutically effective amount of biocompatible polymeric microparticles comprising an angiogenesis inhibitor to the subdural hematoma using targeted endovascular administration. In some embodiments, the angiogenesis inhibitor is a vascular endothelial growth factor A (VEGF-A) antagonist, such as Avastin®.

In some embodiments, the biocompatible polymeric microparticles are administered to the middle meningeal artery. In further embodiments, the subdural hematoma is a chronic subdural hematoma.

In some embodiments, the microparticles comprise a biocompatible polymer that is a biodegradable polymer. In further embodiments the biocompatible polymer is poly-vinyl alcohol. In some embodiments, the microparticles have a diameter from about 40 μm to about 1200 μm. In further embodiments, the microparticles have a diameter from about 45 μm to about 250 μm. In some embodiments, the angiogenesis inhibitor is distributed within the microparticle. In further embodiments the angiogenesis inhibitor is coated or conjugated to the surface of the microparticle.

The invention can include any combination of the features described above.

### BRIEF DESCRIPTION OF THE FIGURES

FIGS. 1A-1C which provide a schematic Illustration of the mechanism of bevacizumab: A) there is a hypervascular tumor surrounded with VEGF protein; B) the bevacizumab compound binds to the free VEGF and reduces the concentration of the free VEGF; C) the reduction of available VEGF results in diminished blood supply to the tumor and tumor shrinkage.

FIG. 2 provides an illustration of middle meningeal artery (MMA) anatomy originated from internal maxillary artery and coursing in the inner skull.

FIG. 3 provide an illustration showing that delivery of embolic particles to a vascular bed leads to arterial occlusion.

### DETAILED DESCRIPTION

The present invention provides a method of treating a solid tumor or a subdural hematoma in a subject in need thereof. The method includes administering a therapeutically effective amount of biocompatible polymeric microparticles comprising an angiogenesis inhibitor to the solid tumor using targeted endovascular administration.

### Definitions

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, and other references mentioned herein are incorporated by reference in their entirety.

As used in the description of the invention and the appended claims, the singular forms “a,” “an,” and “the” are intended to include the plural forms as well, unless the context clearly indicates otherwise. In addition, the recitations of numerical ranges by endpoints include all numbers subsumed within that range (e.g., 1 to 5 includes 1, 1.5, 2, 2.75, 3, 3.80, 4, 5, etc.).

“Treating”, as used herein, means ameliorating the effects of, or delaying, halting or reversing the progress of a disease or disorder. The word encompasses reducing the severity of a symptom of a disease or disorder and/or the frequency of a symptom of a disease or disorder.

The language “effective amount” or “therapeutically effective amount” refers to a nontoxic but sufficient amount of the composition used in the practice of the invention that is effective treat a solid tumor. The desired treatment may be prophylactic and/or therapeutic. That result can be reduction and/or alleviation of the signs, symptoms, or causes of a disease or disorder, or any other desired alteration of a biological system. An appropriate therapeutic amount in any individual case may be determined by one of ordinary skill in the art using routine experimentation.

A “subject”, as used therein, can be a human or non-human animal. Non-human animals include, for example, livestock and pets, such as ovine, bovine, porcine, canine, feline and murine mammals, as well as reptiles, birds and fish. Preferably, the subject is human.

“Pharmaceutically acceptable carrier” refers herein to a composition suitable for delivering an active pharmaceutical ingredient, such as the composition of the present invention, to a subject without excessive toxicity or other complications while maintaining the biological activity of the active pharmaceutical ingredient. Protein-stabilizing excipients, such as mannitol, sucrose, glucose, polysorbate-80 and phosphate buffers, polymers such as polyethylene glycol (PEG), polyvinyl alcohol (PVA), pluronics, are typically found in such carriers, although the carriers should not be construed as being limited only to these compounds.

The term “biodegradable” as used herein refers to a polymer that can be broken down by either chemical or physical process, upon interaction with the physiological environment subsequent to administration, and erodes or dissolves within a period of time, typically within days, weeks or months. A biodegradable material serves a temporary function in the body, and is then degraded or broken into components that are metabolizable or excretable.

“Biocompatible,” as used herein, refers to any material that does not cause injury or death to the animal or induce an adverse reaction in an animal when placed in intimate contact with the animal's tissues. Adverse reactions include for example inflammation, infection, fibrotic tissue formation, cell death, or thrombosis. The terms “biocompatible” and “biocompatibility” when used herein are art-recognized and mean that the referent is neither itself toxic to a host (e.g., an animal or human), nor degrades (if it degrades) at a rate that produces byproducts (e.g., monomeric or oligomeric subunits or other byproducts) at toxic concentrations, does not cause prolonged inflammation or irritation, or does not induce more than a basal immune reaction in the host. It is not necessary that any subject composition have a purity of 100% to be deemed biocompatible. Hence, a subject composition may comprise 99%, 98%, 97%, 96%, 95%, 90% 85%, 80%, 75% or even less of biocompatible agents, e.g., including polymers and other materials and excipients described herein, and still be biocompatible.

### Treatment of Subdural Hematoma

In one aspect, the present invention provides a method of treating a subdural hematoma in a subject in need thereof. The method includes administering a therapeutically effective amount of biocompatible polymeric microparticles comprising an angiogenesis inhibitor to the subdural hematoma using targeted endovascular administration.

A subdural hematoma (SDH) is a type of bleeding in which a collection of blood gathers between the inner layer of the dura mater and the arachnoid mater of the meninges surrounding the brain. It usually results from tears in bridging veins that cross the subdural space. Symptoms of subdural hematomas include seizures, pain, numbness, headache, dizziness, disorientation, amnesia, weakness or lethargy, irritability, loss of consciousness, nausea, and blurred vision. A CT scan or MRI scan can be used to diagnose subdural hematomas.

Subdural hematomas include acute subdural hematomas and chronic subdural hematomas. Acute subdural hematoma is usually caused by external trauma that creates tension in the wall of a bridging vein as it passes between the arachnoid and dural layers of the brain's lining (i.e., the subdural space). Intracerebral hemorrhage and ruptured cortical vessels (blood vessels on the surface of the brain) can also cause an acute subdural hematoma. The signs and symptoms of an acute subdural hematoma can occur immediately, or within minutes, of occurrence.

In some embodiments, the subdural hematoma is a chronic subdural hematoma. Symptoms of chronic SDH are usually delayed by three weeks or more following injury. In chronic subdural hematomas, blood accumulates in the dural space as a result of damage to the dural border cells. The resulting inflammation leads to new membrane formation through fibrosis and produces fragile and leaky blood vessels through angiogenesis, permitting the leakage of red blood cells, white blood cells, and plasma into the hematoma cavity.

The present method includes administering a therapeutically effective amount of biocompatible polymeric microparticles comprising an angiogenesis inhibitor to a subject. Angiogenesis inhibitors are compounds that inhibit the growth of new blood vessels (i.e., angiogenesis). Examples of angiogenesis inhibitors include VEGF antagonists, tyrosine kinase inhibitors, fibroblast growth factor inhibitors, angiopoietins, platelet-derived growth factor inhibitors, hepatocyte growth factor inhibitors, and placental growth factor inhibitors. Lopes-Chelho et al., Int J Mol Sci., 22(7):3765 (2021).

In some embodiments, the angiogenesis inhibitor is a vascular endothelial growth factor A (VEGF-A) antagonist. Strategies to inhibit the VEGF pathway include antibodies directed against VEGF or VEGFR, soluble VEGFR/VEGFR hybrids. Takahashi et al., Biol Pharm Bull., 34(12):1785-8 (2011). Examples of VEGF antagonists include Avastin®, Lucentis®, and Eylea®.

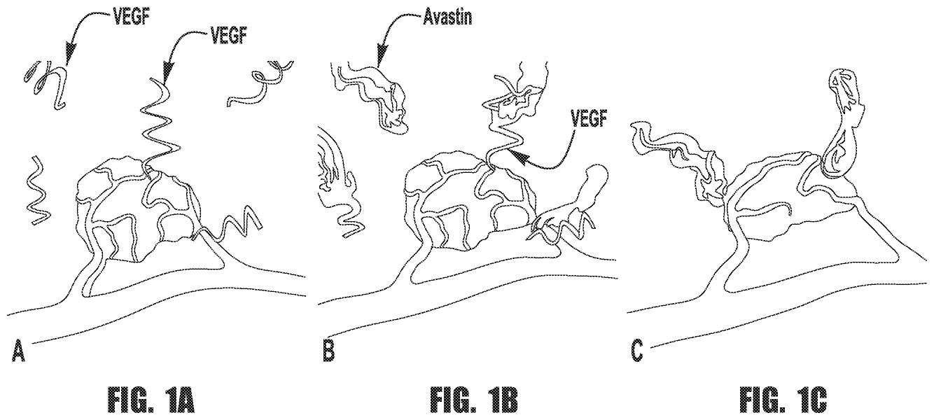

In some embodiments, the angiogenesis inhibitor is Avastin®, or an Avastin® biosimilar compound. Avastin®, also known as Bevacizumab, is recombinant humanized monoclonal antibody that blocks angiogenesis by inhibiting VEGF-A. See FIGS. 1A-1C, which illustrate how Avastin® antagonizes VEGF-A mediated tumor neovascularization, thereby slowing tumor progression, and S. Mukherji, AJNR Am J Neuroradiol., 31(2):235-6 (2010). Bevacizumab was originally derived from a mouse monoclonal antibody generated from mice immunized with the 165-residue form of recombinant human vascular endothelial growth factor. It was humanized by retaining the binding region and replacing the rest with a human full light chain and a human truncated IgG1 heavy chain, with some other substitutions. VEGF-A is a growth factor protein that stimulates angiogenesis in a variety of diseases, including cancer.

The angiogenesis inhibitor is administered using biocompatible polymeric microparticles. Microparticles, as the term is used herein, are particles having a matrix-type structure with a size of 1200 micrometers or less. The microparticles are generally spherical structures, although other shapes such as rods, cubes, cylinders, or amorphous shapes are also possible. In some embodiments, the microparticles have a size of 500 micrometers or less. In some embodiments, the particles have a diameter from 10 micrometers to 1200 micrometers. In other embodiments, the particles have a diameter from 40 micrometers to 1200 micrometers. In other embodiments, the particles have a diameter from 20 micrometers to 800 micrometers. In further embodiments, the particles have a diameter from 40 micrometers to 600 micrometers, while in yet further embodiments the particles have a diameter from 45 micrometers to 250 micrometers. The diameter of the microparticles refers to their mean hydrodynamic diameter. The hydrodynamic diameter can be readily determined using dynamic light scattering (DLS).

The angiogenesis inhibitor can be included in the microparticles in various different ways. For example, the angiogenesis inhibitor can be dispersed or distributed within the microparticle, or it can be encapsulated within the microparticle. In some embodiments, the angiogenesis inhibitor is substantially evenly dispersed within the microparticle. Alternately, or in addition, the angiogenesis inhibitor can be coated on the surface of the microparticle, or conjugated to microparticle surface.

The microparticles of the invention can comprise a wide variety of different types of polymers. Preferably, the microparticle comprises one or more biocompatible polymers. Examples of biocompatible polymers include natural or synthetic polymers such as polystyrene, polylactic acid, polyketal, butadiene styrene, styreneacrylic-vinyl terpolymer, polymethylmethacrylate, polyethylmethacrylate, polyalkylcyanoacrylate, styrene-maleic anhydride copolymer, polyvinyl acetate, polyvinylpyridine, polydivinylbenzene, polybutyleneterephthalate, acrylonitrile, vinylchloride-acrylates, polycaprolactone, poly(alkyl cyanoacrylates), poly(lactic-co-glycolic acid), and the like.

In further embodiments, the microparticle comprises one or more biodegradable polymers. Use of biodegradable polymers provides the advantages of using microparticles that will eventually disintegrate, which can facilitate release of the angiogenesis inhibitor and elimination of the microparticles in vivo. However, angiogenesis inhibitor can also be released from the matrix of non-biodegradable polymers as a result of gradual efflux from channels within the polymer matrix, including those formed by soluble materials included in the polymer matrix.

Examples of biodegradable polymers include polylactide polymers include poly(D, L-lactide)s; poly(lactide-co-glycolide) (PLGA) copolymers; polyglycolide (PGA) and polydioxanone; caprolactone polymers; chitosan; hydroxybutyric acids; polyanhydrides and polyesters; polyphosphazenes; and polyphosphoesters. A preferred biodegradable polymer for use in the microparticles is poly-(DL-lactide-co-glycolide).

Other biodegradable polymers that can be used in the microparticles include AB diblock copolymers such as poly(ethylene glycol) methyl ether-block-poly(D, L-lactide); poly(ethylene glycol) methyl ether-block-poly(lactide-co-glycolide) PEG; poly(ethylene glycol)-block-poly(8-caprolactone) methyl ether PEG; and polypyrrole-block-poly(caprolactone). Further biodegradable polymers include ABA triblock copolymers such as polylactide-block-poly(ethylene glycol)-block-polylactide PLA; poly(lactide-co-glycolide)-block-poly(ethylene glycol)-block-poly(lactide-co-glycolide); poly(lactide-co-caprolactone)-block-poly(ethylene glycol)-block-poly(lactide-co-caprolactone); polycaprolactone-block-polytetrahydrofuran-block-polycaprolactone; and polyglycolide-block-poly(ethylene glycol)-block-polyglycolide PEG.

Another biodegradable polymer that can be used in some embodiments of the invention is an N-alkylacrylamide copolymer. N-alkylacrylamide is a hydrophobic monomer having an alkyl group of C₃ to C₆. For example, in some embodiments, the biodegradable polymer is a copolymer of an N-alkylacrylamide, a vinyl monomer, and a polyethylene glycol (PEG) conjugate. However, in some embodiments, the biodegradable polymer is any of the biodegradable polymers described herein other than a copolymer of an N-alkylacrylamide, a vinyl monomer, and a PEG conjugate. Use of microparticles comprising biodegradable polymers a copolymer of an N-alkylacrylamide, a vinyl monomer, and a PEG conjugate are described in U.S. Pat. No. 9,138,416, the disclosure of which is incorporated herein by reference.

Biodegradable polymers also include various natural polymers. Examples of natural polymers include polypeptides including those modified non-peptide components, such as saccharide chains and lipids; nucleotides; sugar-based biopolymers such as polysaccharides; cellulose; carbohydrates and starches; dextrans; lignins; polyamino acids; adhesion proteins; lipids and phospholipids (e.g., phosphorylcholine).

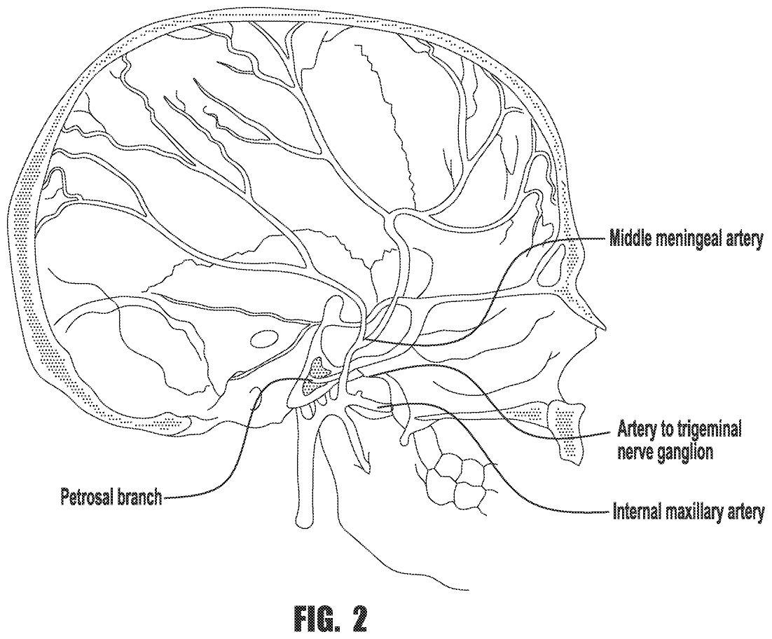

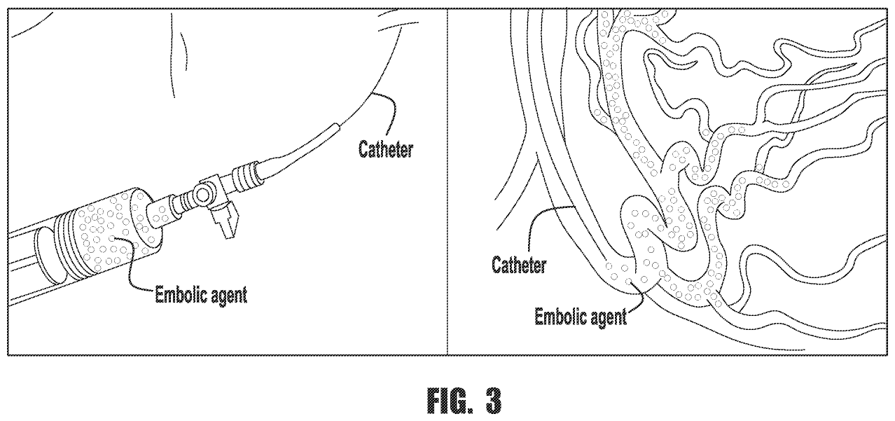

The microparticles including the angiogenesis inhibitor are administered to the subdural hematoma using targeted endovascular administration. By targeted, what is meant is that the microparticles are administered at a location that will result in at least a substantial portion of the angiogenesis inhibitor within the microparticles being delivered to the diseased site. In addition, the microparticles are administered endovascularly. This means that the microparticles are administer to a vascular bed that is in communication with the diseased site. The vascular bed is the part of the vascular system (e.g., blood vessels) associated with a particular organ. For example, when treating a subdural hematoma, the vascular bed can include the middle meningeal artery (MMA). The MMA is a branch of the maxillary artery, which itself is derived from the external carotid artery. It enters the skull through the foramen spinosum, courses through dura and divides into frontal and parietal branches (FIG. 2). The MMA, together with anterior meningeal artery and posterior meningeal artery, supplies the meninges. Moshayedi P. and Libeskind D., Front Neurol., 11:923 (2020). Administration of the microparticles to the vascular bed provides the benefit of blocking blood flow both as a result of the angiogenesis inhibitor and due to physical blockade of the vascular bed by the microparticles, providing an increased and in some cases synergistic effect. FIG. 3 shows how delivery of embolic particles to a vascular bed can lead to arterial occlusion as a result of the particles filling the space within the vascular bed. For a further discussion of endovascular embolization by transcatheter delivery of particles, see Sheth et al., J Funct Biomater, 8(2):12 (2017).

In some embodiments, an additional therapeutic agent is administered to the subject being treating for a subdural hematoma. The additional therapeutic agent can be administered concurrent with or subsequent to the administration of the angiogenesis inhibitor-containing microparticles. The therapeutic agent can be included in the microparticles, or it can be administered separately. In some embodiments, the additional therapeutic agent is angiogenesis inhibitor that is administered in combination with the angiogenesis inhibitor-containing microparticles but using a different method of delivery. For example, angiogenesis inhibitor can be co-administered with angiogenesis inhibitor-containing microparticles in a pharmaceutically acceptable solution or in a gel. Alternately, the additional compound can be another therapeutic agent useful for treating subdural hematoma, such as atorvastatin, dexamethasone, or mannitol.

### Treatment of Solid Tumors

Another aspect of the present invention provides a method of treating a solid tumor in a subject in need thereof. The method includes administering a therapeutically effective amount of biocompatible polymeric microparticles comprising an angiogenesis inhibitor to the solid tumor using targeted endovascular administration.

Cancer is a disease of abnormal and excessive cell proliferation. Cancer is generally initiated by an environmental insult or error in replication that allows a small fraction of cells to escape the normal controls on proliferation and increase their number. The damage or error generally affects the DNA encoding cell cycle checkpoint controls, or related aspects of cell growth control such as tumor suppressor genes. As this fraction of cells proliferates, additional genetic variants may be generated, and if they provide growth advantages, will be selected in an evolutionary fashion. Cells that have developed growth advantages but have not yet become fully cancerous are referred to as precancerous cells. Cancer results in an increased number of cancer cells in a subject.

Cancer cells may form an abnormal mass of cells called a tumor, the cells of which are referred to as tumor cells. The overall amount of tumor cells in the body of a subject is referred to as the tumor load. Tumors can be either benign or malignant. A benign tumor contains cells that are proliferating but remain at a specific site and are often encapsulated. The cells of a malignant tumor, on the other hand, can invade and destroy nearby tissue and spread to other parts of the body through a process referred to as metastasis.

Cancer is generally named based on its tissue of origin. There are several main types of cancer. Carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow and causes large numbers of abnormal blood cells to be produced and enter the bloodstream. Lymphoma and multiple myeloma are cancers that begin in the cells of the immune system. Cancer can also be characterized based on the organ in which it is growing. Examples of cancer characterized in this fashion include bladder cancer, prostate cancer, liver cancer, breast cancer, colon cancer, and leukemia.

A solid tumor is an abnormal mass of tissue that usually does not contain cysts or liquid areas. Solid tumors can be benign or malignant. Different types of solid tumors are named for the type of cells that form them. Examples of solid tumors are sarcomas, carcinomas, and lymphomas. Leukemias (cancers of the blood) generally do not form solid tumors. In some embodiments, the solid tumor being treated is selected from the group of cancer types consisting of breast, colon, bladder, prostate, and lung cancer. In some embodiments, the solid tumor is a brain tumor, while in further embodiments the brain tumor is a meningioma.

In some embodiments, the solid tumor being treated is a brain tumor. Brain tumors include primary tumors, which start within the brain, and secondary tumors, which most commonly have spread from tumors located outside the brain, known as brain metastasis tumors. The most common primary brain tumors include gliomas, meningiomas, pituitary adenomas, and nerve sheath tumors. In some embodiments, the brain tumor being treated is a meningioma. Meningiomas, also known as meningeal tumors, are slow-growing tumors that form in the meninges, which are the outer three layers of tissue between the skull and the brain that cover and protect the brain just under the skull. Brain tumors are typically diagnosed using imaging techniques such as magnetic resonance imaging (MRI) and computed tomography (CT) scans.

Microparticles used for treating a solid tumor are administered using targeted endovascular administration. By targeted, what is meant is that the microparticles are administered at a location that will result in at least a substantial portion of the angiogenesis inhibitor within the microparticles being delivered to the diseased site. Likewise, endovascular administration indicates that the microparticles are administer to a vascular bed that is in communication with the diseased site. In the case of a brain tumor, this can include administering the microparticles to the middle meningeal artery. However, for different types of solid tumors, this will include administering the microparticles to different vascular beds proximal to the solid tumor being treated. As with treatment of subdural hematomas, administration of the microparticles to the vascular bed provides the benefit of blocking blood flow both as a result of the angiogenesis inhibitor and as a result of physical blockage by the microparticles.

A solid tumor can be treated using essentially the same method as that used for the treatment of subdural hematomas described herein. For example, in some embodiments, the angiogenesis inhibitor is a vascular endothelial growth factor A (VEGF-A) antagonist, while in further embodiments. the angiogenesis inhibitor is Avastin®. In some embodiments, the angiogenesis inhibitor is distributed within the microparticle, while in further embodiments the angiogenesis inhibitor is coated or conjugated to the surface of the microparticle.

The biocompatible polymeric microparticles can have any of the microparticle characteristics described herein. In some embodiments, the biocompatible polymer is a biodegradable polymer. In further embodiments, the biocompatible polymer is poly-vinyl alcohol. In further embodiments, the microparticles have a diameter from about 40 μm to about 1200 μm, while in yet further embodiments the microparticles have a diameter from about 45 μm to about 250 μm.

The solid tumor can also be treated using one or more additional methods known to those skilled in the art for treating solid tumors. Other methods of treating a solid tumor include the use of a method selected from the group consisting of cryoablation, thermal ablation, radiotherapy, chemotherapy, radiofrequency ablation, electroporation, alcohol ablation, high intensity focused ultrasound, photodynamic therapy, administration of monoclonal antibodies, and administration of immunotoxins. In some embodiments, the solid tumor is also treated by chemotherapy and/or radiotherapy.

### Formulation and Administration

Forms of microparticle delivery include use of a transluminal local drug delivery device or transendocardial delivery system; intravascular or intra-arterial, cerebrovascular delivery using infusion catheters; periadventitial delivery; perivascular delivery; direct injection into arterial wall; intravenous/intra-arterial injection; intravenous/intra-arterial infusion; localized tissue injection near affected blood vessel; microneedle patches; and administration using a microneedle injection balloon. The type of delivery device used will depend on the location of the solid tumor being treated, although use of a flexible catheter is typical.

In some embodiments, the microparticles are administered as part of a pharmaceutical composition. For example, in some embodiments, a microparticle of the invention maybe combined with a pharmaceutically acceptable vehicle or carrier to provide a pharmaceutical composition. The microparticles may be present in a pharmaceutical composition in an amount from 0.001 to 99.9 wt %, more preferably from about 0.01 to 99 wt %, and even more preferably from 0.1 to 95 wt %. For instance, in embodiments where the microparticles are administration by injection (e.g., intraperitoneally, intravenously, subcutaneously, intramuscularly, etc.), the compositions are preferably combined with pharmaceutically acceptable vehicles such as saline, Ringer's solution, dextrose solution, and the like.

The compositions for administration will commonly comprise a suspension of the microparticles in a pharmaceutically acceptable carrier, preferably an aqueous carrier, which is selected so as not to affect the biological activity of the combination. Examples of such carriers are distilled water, physiological phosphate-buffered saline, Ringer's solutions, dextrose solution, and Hank's solution. These suspensions are sterile and generally free of undesirable matter. These compositions may be sterilized by conventional, well-known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like.

Single or multiple administrations of the compositions may be administered depending on the dosage and frequency as required and tolerated by the subject. In any event, the administration regime should provide a sufficient quantity of the composition of this invention to effectively treat the subject. The formulated microparticles can be administered as a single dose or in multiple doses.

One of skill in the art will recognize that the amount of the microparticles in the formulations can vary widely, and will be selected primarily based on fluid volumes, viscosities, body weight and the like in accordance with the particular mode of administration selected and the subject's needs. In one embodiment, the amount of microparticle administered is between about 0.25 μmol/kg and about 3 μmol/kg equivalent of enzyme. In another embodiment, the amount of microparticle administered is between about 0.5 μmol/kg and about 1.5 mol/kg equivalent of. In yet another embodiment, the amount of microparticle administered is about 1 μmol/kg equivalent of enzymes. In still another embodiment, the amount of microparticle administered is between about 0.1 g/kg and about 0.5 g/kg.

The complete disclosure of all patents, patent applications, and publications, and electronically available materials cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of treating a solid tumor in a subject in need thereof, comprising administering a therapeutically effective amount of biocompatible polymeric microparticles comprising an angiogenesis inhibitor to the solid tumor using targeted endovascular administration.

2. The method of claim 1, wherein the angiogenesis inhibitor is a vascular endothelial growth factor A (VEGF-A) antagonist.

3. The method of claim 1, wherein the angiogenesis inhibitor is Avastin®.

4. The method of claim 1, wherein the biocompatible polymer is a biodegradable polymer.

5. The method of claim 4, wherein the biocompatible polymer is poly-vinyl alcohol.

6. The method of claim 1, wherein the solid tumor is a brain tumor.

7. The method of claim 6, wherein the brain tumor is a meningioma.

8. The method of claim 7, wherein the biocompatible polymeric microparticles are administered to the middle meningeal artery.

9. The method of claim 1, wherein the microparticles have a diameter from about 40 μm to about 1200 μm.

10. The method of claim 1, wherein the microparticles have a diameter from about 45 μm to about 250 μm.

11. The method of claim 1, wherein the angiogenesis inhibitor is distributed within the microparticle.

12. The method of claim 1, wherein the angiogenesis inhibitor is coated or conjugated to the surface of the microparticle.

13. The method of claim 1, wherein the solid tumor is also treated by chemotherapy and/or radiotherapy.

14. A method of treating a subdural hematoma in a subject in need thereof, comprising administering a therapeutically effective amount of biocompatible polymeric microparticles comprising an angiogenesis inhibitor to the subdural hematoma using targeted endovascular administration.

15. The method of claim 14, wherein the angiogenesis inhibitor is a vascular endothelial growth factor A (VEGF-A) antagonist.

16. The method of claim 14, wherein the angiogenesis inhibitor is Avastin®.

17. The method of claim 14, wherein the biocompatible polymer is a biodegradable polymer.

18. The method of claim 17, wherein the biocompatible polymer is poly-vinyl alcohol.

19. The method of claim 14, wherein the biocompatible polymeric microparticles are administered to the middle meningeal artery.

20. The method of claim 14, wherein the microparticles have a diameter from about 40 μm to about 1200 μm.

21. The method of claim 14, wherein the microparticles have a diameter from about 45 μm to about 250 μm.

22. The method of claim 14, wherein the angiogenesis inhibitor is distributed within the microparticle.

23. The method of claim 14, wherein the angiogenesis inhibitor is coated or conjugated to the surface of the microparticle.

24. The method of claim 14, wherein the subdural hematoma is a chronic subdural hematoma.
